(19) United States
(12) Patent Application Publication
(10) Pub. No.: US 2007/0098685 A1
(43) Pub. Date: May 3, 2007

(54) Methods and kits to treat chronic inflammatory immune diseases by administering a proteasome inhibitor and an interleukin 2 receptor agonist

(76) Inventor: Stephen J. Brand, Lincoln, MA (US)

Correspondence Address: Sonia K. Guterman;Lawson & Weitzen, LLP, Suite 345, 88 Black Falcon Ave., Boston, MA, 02210-2481, US

(21) Appl. No.: 11/335,445

(22) Filed: Jan. 19, 2006

Related U.S. Application Data
(60) Provisional application No. 60/645,215, filed on Jan. 19, 2005.

(51) Int. Cl. A61K 38/20; A61K 38/17; A61K 31/69

(52) U.S. Cl. 424/85.2; 514/2; 514/64

(57) ABSTRACT

Methods and kits for treatment of immune diseases with an IL2 receptor agonist and a proteasome inhibitor are provided.

### RELATED APPLICATIONS

This application claims the benefit of U.S. provisional application 60/645,215, filed Jan. 19, 2005, which is hereby incorporated in its entirety by reference herein.

### TECHNICAL FIELD

Methods of treating chronic inflammatory diseases are provided.

### BACKGROUND

Chronic inflammatory diseases include rheumatoid arthritis, Crohn's disease (inflammatory bowel disease (IBD)), psoriasis, multiple sclerosis, systemic lupus erythamatosis (SLE), Type I diabetes, and autoimmune thyroiditis. Many of these diseases are common, for example, in the USA 3-5 million patients have rheumatoid arthritis (RA), 7 million have psoriasis, and 500,000 have Crohn's disease. The medical costs are enormous. In addition, there is the less tangible cost of the pain and suffering caused by these diseases on the patients and the disruption of their lives in society. There is a great unmet medical and social need to find effective ways of treating these diseases. Despite the fact that they do not have a high immediate mortality, the chronic burden of suffering caused by these diseases remains a very important societal and medical problem.

Delayed transplant rejection, similar in pathogenesis to the autoimmune diseases mentioned above, is also an important medical problem that needs improved therapy, both to prevent graft rejection and to reduce the toxicity of current treatments.

### SUMMARY

Accordingly, the invention herein in one embodiment provides a method of treating a mammal suffering from an immune condition, the method involving administering to the mammal a course of treatment comprising a combination of an interleukin 2 receptor agonist and a proteasome inhibitor. Administering to the mammal in one embodiment is measuring remission of the immune condition in the absence of continuous treatment. In a related embodiment, remission of the immune condition in the absence of continuous treatment comprises at least 0.2% of an average lifespan of said mammal. In general, the mammal is a human, although the method is envisioned as applying to other mammalian orders, families, and species such as agricultural, zoological, and experimental animals such as rodents, dogs, cats, goats, sheep, horses.

In certain embodiments, the immune condition is selected from: rheumatoid arthritis, Crohn's disease, psoriasis, and Type I diabetes, although other immune conditions are within the scope of the invention. Alternatively, the immune condition is immune rejection of a transplanted allogenic graft of organ or tissue.

Embodiments of the method include that the course of treatment comprises a period of time less than 3 months, and that the administering is delivering by a route that is systemic, for example, the route of systemic administering is at least one of: oral, subcutaneous, intramuscular and intravenous.

An embodiment of the method further involves observing a temporal sequence in which cellular responses to interleukin 2 and proteasome inhibitor coincide. In an embodiment of the method, administering is performed at a frequency of less than once in two days. Alternatively, administering is performed at a frequency of less than once in seven days. In an embodiment of the method, administering the combination involves administering the agonist and the inhibitor substantially simultaneously. Alternatively, administering the combination is administering the agonist and the inhibitor sequentially. Further, in a related embodiment, the treatment is administering doses of the agonist and the inhibitor at different frequencies, for example, administering the agonist is more frequent than administering the inhibitor.

In certain embodiments, the method further can include comparing administering the combination to administering a control treatment comprising an otherwise identical dose of the inhibitor however absent the agonist, and observing that the dose of the inhibitor absent the agonist is ineffective in treating the mammal for the immune disease. In certain alternative embodiments, the method further can include comparing administering the combination to administering a control treatment comprising an otherwise identical dose of the agonist however absent the inhibitor, and observing that the dose of the agonist absent the inhibitor is ineffective in treating said mammal for the immune disease.

In exemplary embodiments, the agonist is human recombinant interleukin 2, for example, the human recombinant interleukin 2 is Proleukin^{™} des ala-1 serine 125 human interleukin 2. The dose of interleukin 2 receptor agonist the mammal is less than 20 million units, for example, the dose of interleukin 2 receptor agonist to the mammal is less than 7 million units. In exemplary embodiments, the inhibitor is a peptide boronic acid, for example, the peptide boronic acid is PS-341 (Velcade™), Pyz Phe boro leu; Pyz2,5 pyraninecarboxylic acid. The dose of the PS-341 is less than 1 mg per m² of the mammal. In an embodiment of the method, the course of treatment is administering the inhibitor on days 1, 5, 9 and 13 of a 21 day cycle.

Another embodiment of the invention herein is use of a combination of a proteasome inhibitor and an interleukin 2 receptor agonist to induce apoptosis selectively in pathogenic CD4⁺ Th1 T cells. This method can further comprise measuring induction of apoptosis in activated Interleukin 2 responsive CD4⁺ Th1 T cells. Exemplary embodiments include further measuring a decrease of antiapoptotic protection by BCL2 and other antiapoptotic regulatory molecules, and measuring inhibiting of activation of the NFκB transcription factor by proteasome destruction of IκB; further comprising measuring increasing amounts of pro-apoptotic control proteins; further measuring inducing initial cell cycle activation from G₀ to G₁ of cell proliferation by interleukin 2 stimulation, and measuring inhibiting progression from Gₛ-G₁ by proteasome inhibition, for example, measuring increasing pro-apoptotic proteins by prolonging and increasing expression of interleukin-2-activated c-myc levels through preventing c-myc destruction by the proteasome, and through increasing p53 levels by proteasome inhibition both by activating p53 expression through cell cycle arrest and by inhibiting degradation of p53 by the proteasome; further measuring inhibiting NFκB activation and the increasing c-myc and p53 acting in synergy together or in pairs, and measuring initiating pro-apoptotic changes in a plurality apoptosis control proteins, for example, measuring enhancement of potency by intracellular sites of action, and measuring the transcription factors multiple convergent upstream cell signaling pathways controlling apoptosis, or measuring enhancement of potency by intracellular sites of action, the transcription factors that initiate pro-apoptotic changes in apoptosis control proteins, or measuring enhancement of potency by coordinated changes in the activity of multiple transcription factors controlling apoptosis through inhibiting common pathway of proteolysis, by a drug acting on a single defined molecular target, the proteasome.

Another embodiment of the invention herein is a kit for treating an immune condition comprising a unit dose of each of an interleukin 2 receptor agonist, a proteasome inhibitor, and a container. The kit can further include instructions for use. In certain embodiments, the dose is contained in an infusion container.

### BRIEF DESCRIPTION OF THE DRAWINGS

FIG. 1 is a drawing of a protocol for induction and assay of delayed-type hypersensitivity (DTH) in mice, as a function of time from left to right. Groups of experimental animals are sensitized on two successive days (at time points indicated as days—5 and —4 in the Figure) by application of 2,4-dinitro-1-fluorobenzene (DNFB; obtained from Sigma, St. Louis, Mo) to footpads, the DNFB dissolved in acetone or acetone/olive oil. After an additional day (at time indicated as day—3) proteasome inhibitor PS 341 and interleukin 2 (IL 2) are administered by intraperitoneal injection (IP). Animals are challenged, elicited for a T cell response, with DNFB topically applied to a target tissue such as ears at a point in time two days after administration, the point in time indicated as day 0. Swelling of the target, i.e., ear marker organ or tissue is measured at subsequent time points, exemplified as day 1 in the Figure.

FIG. 2 is a bar graph that shows exemplary data following induction of DTH and administration of the combination therapy according to the protocol in FIG. 1, with the exception that PS 341 and IL 2 were administered by subcutaneous injection (SC). Groups of experimental and control mice are administered an IL2 dose which is a constant amount (4 ng) in each group with the exception of a group of control animals not administered IL2, and are administered an amount of PS 341 that is varied (from 0.003 to 0.01 or 0.03 mg/kg total weight) in each of the various experimental groups. A control group of mice is administered IL 2 alone (no PS 341).

### DETAILED DESCRIPTION

In one embodiment, the invention provides a method of treating a mammal suffering from an immune disease, the method comprising administering to the mammal a course of treatment having a combination of an interleukin 2 receptor agonist and a proteasome inhibitor. In a related embodiment, administering to the mammal is further administering the combination by a protocol in the absence of continuous treatment, and thereby remitting the immune disease. For example a period of remitting the immune disease in the absence of continuous treatment comprises at least 0.2% of an average lifespan of said mammal. In general, the mammal is a human.

The immune disease is exemplified but not limited to one or more of rheumatoid arthritis, Crohn's disease, psoriasis, multiple sclerosis and Type I diabetes. In a particular embodiment, the immune disease is rheumatoid arthritis. In another particular embodiment, the immune disease is Crohn's disease. In an alternative embodiment, the immune disease is immune rejection of a transplanted allogenic graft of an organ, a cell or a tissue.

The course of treatment in certain embodiments comprises a period of time that is not a continuous administration, compared to the course of the chronic condition being treated. For example, the course of treatment comprises a period of time less than 3 months. Generally, the route of administering the combination is systemic. For example, the route of systemic administering is at least one of: oral, subcutaneous, intramuscular and intravenous. In a preferred embodiment, the route of administering is intravenous. Further, a protocol of administering is in some embodiments, providing each of the interleukin 2 receptor agonist and the proteasome inhibitor in a temporal sequence wherein the cellular responses to the agonist and to the inhibitor coincide. For example, administering is performed at a frequency of less than once in two days, or at a frequency of less than once in three days, or at a frequency of less than once in seven days. In one embodiment, administering the combination comprises administering the agonist and the inhibitor substantially simultaneously. Alternatively, the combination is administering the agonist and the inhibitor sequentially. The treatment can comprise administering doses of the agonist and the inhibitor at different frequencies. For example, administering the agonist is more frequent than administering the inhibitor. A dose of the proteasome inhibitor in the combination is an amount that generally is ineffective in treating said mammal for the immune disease in the absence of the interleukin-2 agonist. Similarly, a dose of the agonist in the combination is ineffective in treating the mammal for the immune disease in the absence of the inhibitor.

The agonist is exemplified by human recombinant interleukin 2. For example, the human recombinant interleukin 2 is Proleukin™ des ala-1 serine 125 human interleukin 2. In general, the dose of interleukin 2 receptor agonist the mammal is less than 20 million units. For example, the dose of interleukin 2 receptor agonist to the mammal is less than 7 million units. Administering the agonist is, in one embodiment, injecting a bolus intravenously.

The proteasome inhibitor can be selected from but not limited to two classes: irreversible proteasome inhibitors as exemplified by the lactacystin class of proteasome inhibitors or reversible proteasome inhibitors as exemplified by the peptide boronate class. For example, the peptide boronic acid is PS-341 (Velcadem™), which is Pyz Phe boro leu; Pyz2,5 pyraninecarboxylic acid. An alternative proteasome inhibitor is lactacystin which is an irreversible inhibitor of the proteasome inhibiting by forming a covalent bond with the active site of proteolysis.

Further, administering the dose of the PS-341 is administering less than 1 mg per m² of the surface area of the mammal. Administrating the inhibitor is injecting a bolus intravenously. A protocol for the course of treatment is, in one embodiment, administering the inhibitor once every four days, for example, on days 1, 5, 9 and 13 of a 21 day cycle. The inhibitor is in general administered for one or more cycles.

The present invention in another embodiment provides use of a combination of a proteasome inhibitor and an interleukin 2 receptor agonist to induce selective apoptosis of pathogenic CD4⁺ Th1 T cells, the initiating cause of inflammation. The low doses of IL2 administered in the combination restricts IL2 action to activated pathogenic CD4⁺ Th1 T cells which express high affinity IL2 receptors. Thus, combination causes selective apoptosis of pathogenic CD4⁺ Th1 T cell. Thus, administering the combination is further measuring induction of apoptosis in activated CD4⁺ Th1 T cells.

Inhibiting NFκB activation and the increasing c-myc and p53 act in synergy together or in pairs to initiate pro-apoptotic changes in a plurality apoptosis control proteins, measuring any of which is within the scope of the methods and uses herein. Potency of the method is enhanced also by coordinated pro-apoptotic activity of multiple transcription factors including NFκB, c-myc and p53 controlling apoptosis. The shared pro-apoptotic changes in these transcription factors result from the inhibitor decreasing the common pathway of proteolytic removal by the proteasome.

A general method is provided to treat immune diseases by the systemic administration of a combination comprising proteasome inhibitor with interleukin 2 (IL2) causing selective death by apoptosis of activated CD4⁺ Th1 T cells, the cause of immune diseases. The method derives unusual potency due to proteasome inhibition combined with IL2 stimulation causing concurrent increases in p53 and c-myc levels together and inhibition of NFκB activation. These coincident changes in these transcription factors act synergistically to initiate apoptosis of the cells responding to IL2 stimulation, and activate both the intrinsic and extrinsic pathways of apoptosis in CD4⁺ Th1 T cells, the cause of immune diseases.

The method derives unusual potency by acting at the apex of the inflammatory cascade by removing the activated CD4⁺ Th1 T cells that initiate immune disease. Removal of activated CD4⁺ Th1 T cells decreases secretion of Th1 cytokines, TNF and γ interferon which activate tissue inflammation.

The method teaches that activated T cells can be selectively stimulated by low doses of IL2 since only these T cells have high affinity IL2 receptors. Non-activated T cells with low affinity IL2 receptors are not depleted or suppressed, thus preserving T cell immunity and defense against pathogens and nosocomial infections. Furthermore, the method results in long-term remission by deletion of activated T cells and induction tissue tolerance.

A central feature of this method is treating an immune disease, the method comprising systemically administering a combination of a low dose of each of an interleukin 2 (IL2) and a proteasome inhibitor, to induce selective apoptotic death of CD4⁺ Th1 T cells. Exposure of autoimmune T cells to low concentrations of a proteasome inhibitor will change the T cell's response to IL2 stimulation to activation of apoptosis (programmed cell death).

This method answers an important unmet need for treating immune diseases. Existing treatments of immune diseases generally employ drugs that suppress all immune cells non-specifically. Furthermore, existing drug treatments suppress immune inflammation only during the course of drug administration. Once treatment is withdrawn, inflammation recurs, due to reactivation of the T cell's secretion of pro-inflammatory CD4⁺ Th1 cytokines. Many of these drugs also interfere with fundamental metabolic processes within cells and have serious adverse side effects, for example: lymphomas, leukemia, anemia, neutropenia, septicemia, susceptibility to nosocomial infections, and reduced resistance to infections, impaired wound healing (from administering antimitotic drugs such as Immuran), diabetes, hypertension, adrenal suppression, obesity, depression osteoporosis, alopecia, bruising, muscle atrophy (glucocorticoids), hyperlipidemia, and atherosclerosis (from administering rapamycin).

Specific organ toxicity associated with current medications includes: renal failure (Cyclosporin); liver damage (Methotrexate); cystitis (methotrexate); and gastrointestinal ulceration (non steroidal anti-inflammatory drugs (NSAIDs)).

Recently developed anti-inflammatory agents (the expensive and highly potent monoclonal antibodies OKT3, Infliximab and Enbrel™) also have important and, as yet, not fully defined toxicities, such as cytokine release syndrome and lymphoma. Furthermore, antibody molecules as drugs have major drawbacks: because of their high molecular weight they have difficulty penetrating into joints and poorly vascularized tissues.

The method is greatly superior to immune tolerance therapies which inhibit autoimmune inflammation by inducing immune tolerance by immunization with the specific initiating autoantigen. However immune tolerance therapy has been unsuccessful in treating chronic immune diseases. Practically, immunization can only begin after the onset of the autoimmune disease. During this time the established tissue inflammation has recruited additional antigens, exposed by inflammatory tissue destruction, a phenomenon termed “epitope spreading.” Since many undefined tissue antigens contribute to established chronic inflammatory disease, tolerance therapies using a manageable number of antigens have failed to show efficacy.

A feature of this method provides a new therapy that meets the unmet medical need for improved treatment of immune diseases. This method has the following advantages: improved potency with more profound resolution of inflammation; long-term remission of disease without continuous maintenance immunosuppression; minimal suppression of immune defense to pathogens and nosocomial infections from commensal organisms; lower systemic toxicity than that associated with long-term glucocorticoid treatment; absent or diminished risk of inducing neoplasia.

An aspect of the present invention is a general method to treat most immune diseases by removing the common cause of these diseases, the activated autoimmune T cell. Most immune diseases have a common pathogenesis, the CD4⁺ Th1 T cells which secrete the inflammatory cytokines tumor necrosing factor (TNF) and γ interferon. The present method provides a treatment that selectively kills activated CD4⁺ Th1 T cells, achieving potent treatment of immune diseases with reduced toxicity and without non-specific immune suppression

Another embodiment of the invention is a method for inducing tissue tolerance to autoimmune antigens by expanding the population of CD4⁺ Th2 T cells that are resistant to apoptosis. Although responsive to IL2 , CD4⁺ Th2 cells are resistant to apoptosis because they secrete the cytokine TGFβ, which protects Th2 T cells from apoptosis. Consequently, treatment with the IL2 /proteasome inhibitor combination will result in CD4⁺ Th2 T cells becoming the predominant T cell population in tissues affected by immune disease. Predominance of CD4⁺ Th2 T cells will cause immune tolerance to autoantigens, resulting in long-term remission of disease without maintenance immunosuppression therapy.

A preferred embodiment of the invention is a method of administering systemically a low dose of the IL2 receptor ligand Proleukin™ (Chiron), the FDA approved form of IL2 . Proleukin is a modified recombinant 15300 MW protein analog of human IL2 (des ala-1 serine 125 human IL2 ) made by expression in E. coli. Other IL2 cogeners are also included as effective T cell IL2 receptor agonists as described in U.S. Pat. No. 6,190,656, which is incorporated herein by reference in its entirety. Also methods of IL2 production and formulation are included herein by reference to U.S. Pat. No. 6,190,656.

An embodiment of the invention is a method of administering systemically a low dose of IL2 , thereby diminishing incidence of adverse events. Low doses of IL2 (<20 million Units) are well tolerated after repeated daily dosing-for many weeks. See (Lane, et al. 1997 U.S. Pat. No. 6,190,656 pp. 1-32).

A preferred embodiment of the invention is a method of administering an IL2 dose between 1-10 million units intravenously, thereby achieving blood levels of IL2 in the ng/ml range. (Lane, Kovacs, and Fauci pp. 1-32). These blood levels will result in effective occupation of the high affinity IL2 receptor (K_{d} 10⁻¹¹), on activated T cells, to elicit the IL2 receptor mediated cellular responses described above. Effective T cell stimulation by IL2 can be evaluated by measuring serum levels of IL2R α protein, which is shed following stimulating T cell by IL2 . (Lane, Kovacs, and Fauci pp. 1-32). This provides an important test to determine effective pharmacological response to IL2.

A preferred embodiment of a method of IL2 systemic administration is, without limitation, dosing twice daily by intravenous bolus or by continuous intravenous infusion. Intravenous administration of IL2 for extended periods is well tolerated.

An important embodiment of this method is inhibiting the proteasome, which, in combination with administering low doses of IL2 , elicits a potent apoptotic response in activated T cells. Many IL2 stimulated molecular signaling responses in T cells depend on proteolytic degradation by the proteasome pathway of intracellular proteolysis. The proteasome pathway is important in regulating many cell functions through ensuring rapid removal of short-lived regulatory proteins, including transcription factors such as c-myc and p53 , and the cell cycle control proteins cyclins and p27. The Ub proteasome pathway also can prevent activation of transcription factors that inhibit regulatory proteins by degrading inhibitor proteins, e.g., degrading IκB to activate NFκB. (Hershko 1997 Curr. Opin. Cell Biol. 9 pp. 788-99).

An embodiment of this method is administering systemically a proteasome inhibitor in combination with IL2 to induce T cell apoptosis. Two classes of specific inhibitors of the proteasome have been described: Lactacystin and dipeptide boronates. Lactacystin irreversibly binds to the subunit X proteasome active site N-terminal threonine. (Dick et al., 1997 J. Biol. Chem. 272 pp. 182-88); and (Fenteany and Schreiber 1998 J. Biol. Chem. 273, pp. 8545-48). Dipeptide boronate derivatives are highly potent and specific proteasome inhibitors due to the high affinity and greater stability of the boron-threonine bond formed at the active site of the proteasome. (Adams and Stein, 1996, in “Novel Inhibitors of the Proteasome and Their Therapeutic Use in Inflammation”, Academic Press, Inc., pp. 279-88); and (Adams et al., 1998, U.S. Pat. No. 5,780,454, pp. 1-66). The dipeptide boronates demonstrate also a high degree of enzyme selectivity and are inactive against other proteases including trypsin, chymotrypsin and the cysteine proteases papain, calpain I, calpain II, and cathepsin B. (Adams and Stein pp. 279-88).

A preferred embodiment of this method is administering systemically a boronate proteasome inhibitor in combination with IL2 . Boronate proteasome inhibitors are active in cell based assays and bioavailable after systemic administration, and show efficacy in vivo in experimental cancer and inflammation animal models. (Adams and Stein pp. 279-88); (Conner et al. 1997 J. Pharmacol. & Exptl. Ther. 282 pp. 1615-22); and (Palombella et al. 1998 Proc. Natl. Acad. Sci. U.S. 95 pp.15671-76). Safety is an important issue with systemic administration of proteasome inhibitors, given the plurality of proteins degraded by the proteasome, including many that have vital regulatory functions. While inhibition of the proteasome was once thought to cause major cellular toxicity which would preclude its therapeutic use, this concern has not been confirmed experimentally. Microarray analysis showed that cells treated with therapeutic concentrations of a boronate proteasome inhibitor (Velcade™) altered only a restricted number of proteins. These proteins include short-lived regulatory proteins that principally control growth factor response and cell proliferation as well as heat shock proteins. (Mitsiades et al. 2002 Proc. Natl. Acad. Sci. USA 99 pp. 14373-79). Furthermore, reproducible and safe dosing in animal models and in human subjects has been shown with doses of the boronate proteasome inhibitor PS-341, which has been free from substantial toxicity that would preclude use as a drug from chronic administration, and are therapeutically efficacious. (Orlowski et al. 2002 J. Clin. Oncol. 20 pp. 4420-27); and (Lightcap et al. 2000 Clin. Chem. 46 pp. 673-83).

A preferred embodiment of the method is administering systemically the boronate proteasome inhibitor Velca.de™ (MG-341, PS-341, Pyz Phe boro leu; Pyz2,5 pyraninecarboxylic acid); (Adams et al., U.S. Pat. No. 5,780,454 pp. 1-66). Velcade™ is the first proteasome inhibitor to receive FDA approval, and is a new class of cancer chemotherapy that kills cancer cells by apoptosis. (Mitsiades et al. 2002 Proc. Natl. Acad. Sci. USA 99 pp. 14373-79), (Hideshima et al. 2003 Blood 101 pp. 1530-34). Velcade™ is administered by intravenous bolus injection. (Orlowski et al. 2002 J. Clin. Oncol. 20 pp. is 4420-27); and (Lightcap et al. 2000 Clin. Chem. 46 pp. 673-83). Pharmokinetic analysis of Velcade™ blood concentrations is of little value for monitoring dosing, due to rapid distribution of Velcade™ from the plasma circulation. (Lightcap et al. 2000 Clin. Chem. 46 pp. 67.3-83). Velcade™ levels are monitored by pharmacodynamic measurement of inhibition of proteasome enzyme activity in a blood cell following a bolus intravenous injection; (Lightcap et al. 2000 Clin. Chem. 46 pp. 673-83). One hour after intravenous bolus dosing, maximal blood cell proteasome (80%) inhibition is achieved with a dose of 0.3 mg/kg in rats; (Lightcap et al. 2000 Clin. Chem. 46 pp. 673-83), or a dose of 1.38 mg/m² in humans, (Orlowski et al. 2002 J. Clin. Oncol. 20 pp. 4420-27). In humans, Velcade™ has a long half-life of 24 hours, and is administered once every 4 days to prevent accumulation. (Orlowski et al. 2002 J. Clin. Oncol. 20 pp. 4420-27). There is a quantitatively proportional dose/response relationship of Velcade™ after intravenous bolus injection in both rats and man. In human studies, adverse events are observed at doses that are 1 mg/m² (proteasome inhibition), doses which are considered tolerable only for life threatening diseases such as cancer. However, to be sufficiently well tolerated for treatment of a non-fatal disease such as an immune disease, doses of Velcade™ lower than 1 mg/m² are required.

Without being limited by any particular theory or mechanism, a preferred embodiment of the method is administering systemically a dose of a proteasome inhibitor that gives partial inhibition (20-30%) of red blood cell proteasome activity. In human subjects, a Velcade™ PS-341dose of 0.4 mg/m² gave 30% inhibition of blood cell proteasome activity after a single intravenous bolus injection; (Orlowski et al. 2002 J. Clin. Oncol. 20 pp. 4420-27). No adverse events were observed at doses less than or equal to 0.4 mg/m² after multiple administration. (Orlowski et al. 2002 J. Clin. Oncol. 20 pp. 4420-27) reported also that no significant therapeutic efficacy was seen at this dose. Equivalent partial proteasome inhibition in rats (20-30%) was achieved with a 0.03 mg/kg intravenous bolus dose. In animal models of immune disease, the 0.03 mg/kg dose was well tolerated but also was not effective therapeutically.

An embodiment of the method is administering systemically a combination of doses of each of IL2 and a proteasome inhibitor, the latter of which if administered alone would not cause significant T cell apoptosis. IL2 stimulation of the cell cycle is required for activation of apoptosis by proteasome inhibitors. The dose of proteasome inhibitor PS-341 (Velcade™) that causes partial inhibition of blood proteasome activity does not cause serious adverse events in human subjects.

A feature of the methods herein is that cellular responses to IL2 and PS-341 are contemporaneous for induction of T cell apoptosis. A consequence of this feature is that PS-341 is administered-less frequently than IL2 . The half-life of PS-341 is approximately 24 hours. Therefore, to prevent accumulation, PS-341 is administered systemically in the methods herein once every four days by intravenous bolus injection. By contrast, the half-life of IL2 Proleukin is 1.5 hours, resulting in once or twice daily administration of IL2 , in order to achieve exposure of cells to appropriate amounts of both IL2 and PS-341 for significant periods.

Inhibition of the proteasome by proteasome inhibitors such as PS-341 (Velcade™) blocks activation of NFκB after IL2 stimulation. Consequently, such inhibition by IL2 stimulation removes a key mechanism of antiapoptotic protection. Another feature of the methods herein is the resulting prolonged increase in c-myc expression, which promotes apoptosis after administering systemically the combination of a proteasome inhibitor and IL2. An early event after IL2 stimulation of activated T cells is the expression of the transcription factor, c-myc. IL2 stimulation generally provides only a brief increase in c-myc expression due to rapid proteolysis by the Ub proteasome pathway.

However, when IL2 stimulation is combined with administering a proteasome inhibitor, expression of c-myc is prolonged, which changes the cellular response to activation of apoptosis.

Pharmacological Compositions

The present invention in various embodiments provides methods of administering pharmaceutical compositions comprising a therapeutically effective amount of a combination comprising two agents, an interleukin 2 receptor agonist and a proteasome inhibitor composition as described herein. All of the pharmaceutical compositions described herein can be formulated with or without an agent for immune suppression, and with or without components or devices for sustained release, for delivery locally or systemically. A pharmaceutically acceptable carrier or excipient can be added. Such a carrier includes but is not limited to saline, buffered saline, dextrose, water, glycerol, ethanol, and combinations thereof. The formulation should suit the mode of administration. An “effective amount” as the term is used herein is an amount of the combination of the agents sufficient to achieve a recognized medical endpoint, in this case, remediation of a symptom of a chronic immune or inflammatory disease. The effective amount can be determined empirically by a skilled artisan according to established methods of measurement of relevant parameters, as described herein.

The compositions herein can further comprise wetting or emulsifying agents, or pH buffering agents. The composition can be a liquid solution, suspension, emulsion, tablet, pill, capsule, sustained release formulation, or powder. The compositions can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g., encapsulation in liposomes, microparticles, microcapsules and the like.

In an exemplary embodiment, a composition herein is formulated in accordance with routine procedures as a pharmaceutical composition adapted, for example, for bolus intravenous administration to human beings. Typically, compositions for intravenous administration are solutions in sterile isotonic aqueous buffer. Where necessary, the composition may also include a solubilizing agent and a local anesthetic to ameliorate pain at the site of the injection. Generally, the ingredients are provided either separately or mixed together in unit dosage form, for example, as a dry, lyophilized powder or water-free concentrate in a hermetically sealed container such as an ampoule or sachette, for example, indicating the quantity of active agent. Where the composition is to be administered by intravenous administration by infusion, it can be dispensed with an infusion bottle containing sterile pharmaceutical grade water, buffer, or saline. Where the composition is administered by injection, an ampoule of sterile water or saline for injection can be provided so that the ingredients may be mixed prior to administration. The compositions herein can in various components thereof be formulated as suppositories, which contain active ingredient in the range of about 0.5% to about 10% by weight; oral formulations preferably contain about 10% to about 95% active ingredient by weight. A daily dose is administered as a single dose, or is divided into a plurality of smaller fractional doses, to be administered several times during the day.

As used herein, a dosing schedule refers to a protocol for administering any of the compositions comprising for instance one or more of an in vivo proteasome inhibitor composition as described herein, in an effective dose, administered simultaneously or within a particular interval of each other, for example, within one day of each other, or as a combined preparation, or separately, and includes the amount of the composition delivered per unit time, such as per day, and the duration or period of time over which each composition is administered.

In one aspect, the invention provides a method for preventing or treating an immune or inflammatory disease, the method comprising administering to a mammal in need thereof composition of at least one interleukin 2 receptor agonist and at least one proteasome inhibitor as described herein, each in an amount sufficient to increase the parameters described herein in the mammal; and determining the amount of for example, apoptosis of a set of CD4 cells, thereby treating or preventing the immune disorder. Administering the composition described herein reduces proteasomal activity increases specific cell apoptosis, compared to proteasomal activity or specific cell apoptosis assayed prior to administering the composition, for example, administering the composition reduces proteasomal activity by about 50%, or by about 70%, compared to proteasomal activity assayed prior to administering the composition. One or more parameter that measures an amount of proteasomal activity indicates a reduced activity, compared to proteasomal activity in the mammal assayed prior to administering the composition.

The compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances. Routine determinations of sample levels of a proteasome activity are determined by one of ordinary skill in the art. Effective doses may be extrapolated from dose-response curves derived from in vitro or animal model test systems, by one of ordinary skill in the art of pharmacology. Dosages of the compositions to be administered to a subject are adjusted for known variations from species to species using standard data encompassing criteria for absorption, distribution, half-life kinetics in circulation, metabolism, excretion, and toxicology of the receptor ligands of the embodiments herein. Suitable dosage ranges for administration are generally about 0.01 micrograms to about 10,000 micrograms of each active compound per kilogram body weight per day, for example, about 0.01 micrograms to about 1 microgram/kg, about 0.1 micrograms/kg to about 10 micrograms/kg, about 1 microgram/kg to about 500 micrograms/kg, or about 10 micrograms/kg to about 10 mg/kg of body weight per day. Suitable dosage ranges for administration are thus generally about 0.01 micrograms/kg body weight/day to about 10 mg/kg body weight/day. More accurate dosages can be calculated on the basis of subject surface area, as measured in m² as is known to one of skill in the art of pharmacology.

The invention in other embodiments provides a pharmaceutical pack or kit comprising one or more containers filled with one or more of the ingredients of the pharmaceutical compositions of the invention. In such a pack or kit can be found a container having a unit dosage of each of the interleukin 2 receptor agonist and the proteasome inhibitor. Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

Unless otherwise defined, all technical and scientific terms herein have the same meaning as commonly understood by one of ordinary skill in the art to which this invention pertains. Methods and materials similar or equivalent to those described herein can be used in the practice of the present invention. The invention in various embodiments now having been fully described, additional embodiments are exemplified by the following claims, which are not intended to be construed as further limiting. The contents of all cited references are hereby incorporated by reference in their entirety herein.

### EXAMPLES

IL2 potentiates PS 341 Proteasome Inhibitor Inhibition of Delayed Type Hypersensitivity Reaction in Mice

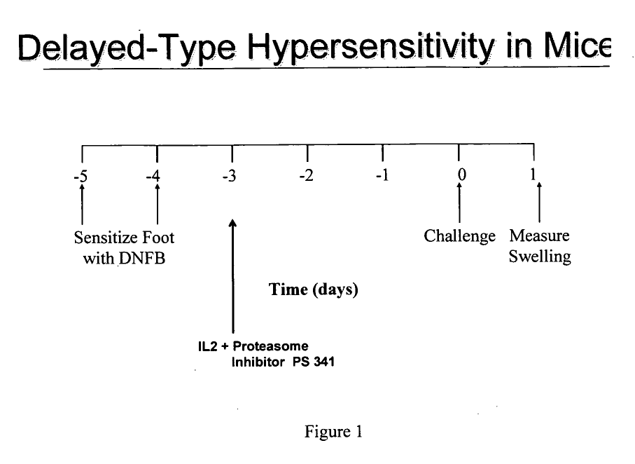

Delayed Type Hypersensitivity reaction in mice is a well-established animal model of CD4Th1 T cell mediated inflammation. DTH was performed according to methods described in Brand et al., Gastroenterology 1999. 116:865-873, and as shown in the protocol in FIG. 1, with the following variation. PS 341 and IL2 were injected by intraperitioneal injection 24 hours after the second DNFB sensitization to the foot pads. At this time IL2 dependent T cell activation to the TNBS antigen occurs.

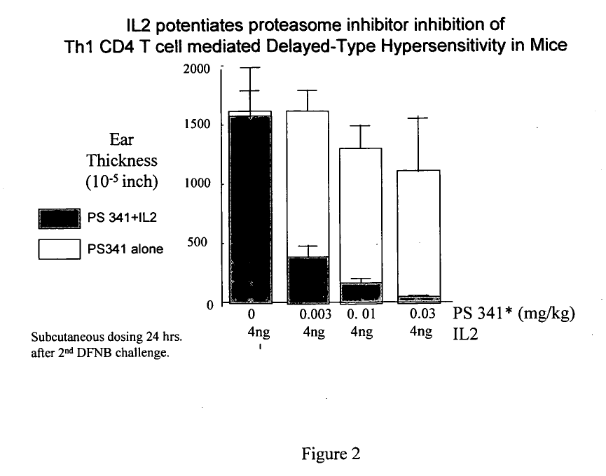

Inflammation was measured by ear swelling after applying topical DNFB. Exemplary results are shown in FIG. 2, in which increasing quantities of PS 341 co-administered with IL2 result in increasing inhibition of DTH. FIG. 2 suggests that co-administration of an interleukin 2 receptor agonist and a proteasome inhibitor are the basis of therapeutic methods and compositions for treating an immune disorder. Further, it is possible that doses over an order of magnitude range of PS 341 administered to an animal, along with an unvarying amount of an interleukin 2 receptor agonist, can produce beneficial effects.

What is claimed is:

1. A method of treating a mammal suffering from an immune condition, comprising administering to the mammal a course of treatment comprising a combination of an interleukin 2 receptor agonist and a proteasome inhibitor.

2. The method of claim 1, wherein administering to the mammal further comprises measuring remission of the immune condition in the absence of continuous treatment.

3. The method of claim 2, wherein remission of the immune condition in the absence of continuous treatment comprises at least 0.2% of an average lifespan of said mammal.

4. The method of claim 1, wherein the mammal is a human.

5. The method of claim 1, wherein the immune condition is selected from: rheumatoid arthritis, Crohn's disease, multiple sclerosis, psoriasis, and Type I diabetes.

6. The method of claim 1, wherein the immune condition is immune rejection of a transplanted allogenic graft of organ or tissue.

7. The method of claim 1, wherein the course of treatment comprises a period of time less than 6 months.

8. The method of claim 1, wherein the administering is delivering by a route that is systemic.

9. The method of claim 8, wherein the route of systemic administering is at least one of: oral, subcutaneous, intramuscular and intravenous.

10. The method of claim 1, wherein administering the combination is administering the agonist and the inhibitor sequentially.

11. The method of claim 1, wherein the treatment is administering doses of the agonist and the inhibitor at different frequencies.

12. The method of claim 11, wherein administering the agonist is more frequent than administering the inhibitor.

13. The method of claim 1 wherein the agonist is human recombinant interleukin 2.

14. The method of claim 13 wherein the human recombinant interleukin 2 is Proleukin™ des ala-1 serine 125 human interleukin 2.

15. The method of claim 13 wherein the dose of interleukin 2 receptor agonist the mammal is less than 20 million units.

16. The method of claim 13 wherein the dose of interleukin 2 receptor agonist to the mammal is less than 7 million units.

17. The method of claim 1, wherein the inhibitor is a peptide boronic acid.

18. The method of claim 17 wherein the peptide boronic acid is PS-341 (Velcade™), Pyz Phe boro leu; Pyz2,5 pyraninecarboxylic acid.

19. The method of claim 18, wherein the dose of the PS-341 is less than 1 mg per m² of the mammal.

20. The method of claim 1, wherein the course of treatment is administering the inhibitor on days 1, 5, 9 and 13 of a 21 day cycle.

21. A use of a combination of a proteasome inhibitor and an interleukin 2 receptor agonist to induce apoptosis selectively in pathogenic CD4⁺ Th1 T cells.

22. The use of claim 21, further comprising measuring inhibiting NFκB activation and the increasing c-myc and p53 acting in synergy together or in pairs, and measuring initiating pro-apoptotic changes in a plurality apoptosis control proteins.

23. The use of claim 22, further comprising measuring enhancement of apoptotic potency by coordinated changes in the activity of multiple transcription factors controlling apoptosis through inhibiting common pathway of proteolysis, by a drug acting on a single defined molecular target, the proteasome.

24. A kit for treating an immune condition comprising a unit dose of each of an interleukin 2 receptor agonist, a proteasome inhibitor, and a container.

25. The kit of claim 24, comprising instructions for use.

26. The kit of claim 24, wherein the dose is contained in an infusion container.
